(19) United States
(12) Patent Application Publication
(10) Pub. No.: US 2010/0099199 A1
(43) Pub. Date: Apr. 22, 2010

(54) METHOD AND APPARATUS FOR ASCERTAINING REAGENT QUALITY

(75) Inventors: Markus Egle, Ladenburg (DE); Robert Gropp, Hochdorf-Assenheim (DE); Stefan Künkel, Karlsruhe (DE); Karl-Heinz Westerhoff, Eppingen-Elsenz (DE)

Correspondence Address: ALEXANDER R SCHLEE;SCHLEE IP INTERNATIONAL P.C., 3770 HIGHLAND AVENUE, SUITE 203, MANHATTAN BEACH, CA, 90266, US

(73) Assignee: LEICA BIOSYSTEMS NUSSLOCH GMBH, Nussloch (DE)

(21) Appl. No.: 12/580,203

(22) Filed: Oct. 15, 2009

(30) Foreign Application Priority Data
Oct. 22, 2008 (DE) 10 2008 052 658.4
Nov. 10, 2008 (DE) 10 2008 056 583.0

(51) Int. Cl. G01N 1/00; C12M 1/34

(52) U.S. Cl. 436/174; 435/287.1

(57) ABSTRACT

A method for determining reagent quality in a device having multiple treatment stations for the treatment of at least one of cytological and histological prepared specimens and an apparatus for performing this method are described. The method comprises providing a carrier element that comprises at least one test material; transferring and treating the carrier element having test material according to a predefined sequence in a plurality of the treatment stations together with the specimens; and evaluating the test material by means of an evaluation device after treatment in the last treatment station in sequence. The described method and apparatus achieve that reagent quality can be determined automatically and the reagents can be replaced at an optimum point in time.

### CROSS-REFERENCE TO RELATED APPLICATION

This application claims the priority of the German patent application DE 102008052658.4 having a filing date of Oct. 22, 2008 and of the German patent application DE 102008056583.0 having a filing date of Nov. 10, 2008. The entire content of these two prior German patent applications is herewith incorporated by reference.

### BACKGROUND OF THE INVENTION

The invention relates to a method and an apparatus for ascertaining reagent quality in the context of units having multiple treatment stations for the treatment of specimens, in particular of cytological and histological prepared specimens.

Cytological and histological methods are used for the investigation of cells that have been obtained, for example, by way of a smear, or from surgical preparations, biopsy material, or other tissue samples. An analysis of the prepared specimens is performed usually for diagnostic purposes in medicine. The preparation, processing, and staining of such prepared specimens is associated, in most cases, with complex processing of the material using a plurality of reagents. Techniques for identifying tissue that has been modified as a result of illness, for example, require an entire series of steps. These include producing a tissue section, encompassing fixing, dehydration, embedding, and sectioning of the tissue, as well as deparaffination, rehydration, and staining of tissue sections and coverslipping of the completed prepared specimens. Typical reagents that are utilized are formalin, butanol, xylene, and paraffin. Dyes such as hematoxylin, methylene blue, Azur, cresyl violet, toluidine blue, Alcian blue, eosin, azocarmine, acid fuchsin, Ponceau, Orange G, picric acid, or Schiff reagent are also used. Also utilized, for the identification and histological localization of substances, are antibodies that are responsible for an antigen-antibody reaction. The antibodies can in turn be detected directly or indirectly by way of certain color reactions in the prepared specimen.

In the context of units that serve for the processing or cells, tissues, or organs and/or for the staining thereof, cytological and histological prepared specimens are delivered to the respectively necessary treatment stations by means of a specimen slide or a basket, and if applicable in transport magazines for the reception of specimen slides and baskets. The treatment stations are loaded with the different reagents. The result of the processing or staining depends critically on the quality of these reagents in the process stations. That quality is highest when the reagents have just been introduced into the treatment stations. The quality of the reagents decreases over time, however, as a result of various factors. In addition to consumption of the reagents by reaction with the target molecules contained in the cytological and histological preparations, these factors also include quality loss due to oxidation of the reagents in air, and due to the carryover of reagents adhering to prepared specimens from one treatment station into another. As a consequence thereof, the reagents must constantly be replenished.

Heretofore either the point in time for a reagent change was determined by the user by means of a visual check of the completed prepared specimens, or the reagents were replaced after a specific, defined time in the treatment station. With the first method, the result of the processing or staining was monitored by the user by simply observing the processed or stained specimens. An assessment is made on the basis of experience. A disadvantage of this procedure is the enormous expenditure of time required for inspection of each individual specimen. In addition, the assessment is made on the basis of the observer's subjective perception, which varies from user to user and is moreover also dependent on the observer's mood. With the second method, replacement of the reagents is accomplished after a predefined period of time has elapsed, or after throughput of a specified number of baskets or transport magazines that have passed through the treatment stations. A disadvantage of this method is that the actual throughput of prepared specimens is not taken into account. For this reason, it is possible for the reagents to be replaced even though a large number of prepared specimens could still have been treated with them. This is particularly disadvantageous specifically with regard to expensive reagents and the environmental impact of toxic, environmentally damaging reagents. Excessively long and frequent use of the reagents, on the other hand, results in insufficient quality in the processed specimens. This is particularly critical because the specimens are often present only in a very small quantity, or in fact involve individual samples. In some circumstances, processing the prepared specimens using lower-quality reagents results in the irrecoverable loss of extremely important information.

### SUMMARY OF THE INVENTION

It is therefore an object of the invention to make available a method in which, in the context of units having multiple treatment stations for the treatment of specimens, in particular of cytological and histological prepared specimens, reagent quality can be ascertained automatically, and in which replacement of the reagents at an optimum point in time is thus made possible. A further object of the invention is to make available a corresponding apparatus with which reagent quality can be automatically ascertained.

The objects are achieved by a method for determining reagent quality in a device having multiple treatment stations for the treatment of at least one of cytological and histological prepared specimens, the method comprising: a) providing a carrier element that comprises at least one test material; b) transferring and treating the carrier element having test material according to a predefined sequence in a plurality of the treatment stations together with the specimens; and c) evaluating the test material by means of an evaluation device after treatment in the last treatment station in sequence.

The respective apparatus for determining reagent quality in a device having multiple treatment stations for the treatment of at least one of cytological and histological prepared specimens comprises a carrier element, at least one test material carried by on the carrier element, and an evaluation device for evaluating the test material.

The carrier element having test material is conveyed through the respective treatment stations concurrently with the specimens. This causes the test material to be subjected to the same conditions as, and to experience treatment steps identical to, the specimens in the treatment stations. While the carrier element having the test material is passing through all the treatment stations that the specimens pass through, the test material reacts with the reagent or reagents of the treatment stations, forming at least one detectable product. The evaluation device senses the detectable product after the last treatment station. Equipping a carrier element with multiple test materials offers the capability, in this context, of carrying out different detection methods in parallel. For this purpose the test materials can be distributed arbitrarily over the carrier element, or they are located in specific regions on the carrier element. What is important in the context of the use of multiple different test materials on one carrier element is that the different test materials treated in the treatment stations supply signals that are distinguishable from one another.

### DETAILED DESCRIPTION OF THE INVENTION

According to an advantageous embodiment of the invention, in the method, a reference carrier element having a test material is previously treated in the reagents of the treatment stations in the predefined sequence. The treatment of the reference carrier element can occur either together with specimens to be treated, or separately, without specimens, in the treatment stations according to the specimen treatment sequence. The test material of the reference carrier element is, as a rule, a test material identical to the test material in step a). It is also conceivable, however, to use different test materials if their characteristic properties supply comparable data after passing through the treatment stations.

After the last treatment station, the characteristic properties of the test material of the reference carrier element that are caused by the treatment are sensed, and are stored as reference data. If the reference carrier having the test material is treated in the treatment stations after a reagent change, that treatment then supplies reference data that were generated with reagents of optimum quality. The characteristic properties of the test material after treatment have a specific, defined relationship to reagent quality. This ensures that the characteristic properties of the test material reflect the quality of the reagent or reagents. The reference carrier element having the test material can, of course, be used not only after replenishment of the reagents. Treatment of the reference carrier element in the treatment stations can be made dependent on the state of the reagents that is to serve as a reference point. It is therefore useful to utilize and evaluate the reference carrier element having test material in the context of reagents that exhibit good quality.

The reference data obtained from the evaluation of the test material serve for evaluation of the carrier elements having test material that are used subsequently.

According to a further advantageous embodiment of the invention, for evaluation of a test material in accordance with step c), the characteristic properties of the test material that are caused by the treatment are compared with the reference data. An evaluation of absolute data can be accomplished for this comparison. Said data are obtained in the same manner from the test material of the reference carrier element and from the test material of the carrier element. It is also possible, however, to identify absolute data only for the test material of the reference carrier element. Quantitative evaluation of the test material of a carrier element is omitted. The evaluation of the carrier elements, and the comparison of the characteristic properties of the test material of the carrier elements with the reference data, then take place purely qualitatively.

According to a further advantageous embodiment of the invention, at least one threshold value is predefined and is stored in the evaluation device. For evaluation of a test material in accordance with step c), a check is made as to whether a conformity exists between the characteristic properties of the test material and the reference data, in consideration of the threshold value. In such a case the quality of the reagents is accepted even if the characteristic properties do not exactly conform to the reference data, but even if they deviate upward or downward by a predefined value from the reference data and are thus within the limits defined by the threshold value.

According to a further advantageous embodiment of the invention, the user is informed when a conformity does not exist between the characteristic properties of the test material and the reference data, in consideration of the threshold value. In the absence of a conformity, it is assumed that the quality of the reagents of the treatment stations is no longer sufficient to achieve the desired or necessary effect in the context of the specimens. The user is therefore made aware of the inadequate quality of the reagents. This is accomplished, for example, by way of an optical or acoustic signal, or by means of an indication on a screen of the apparatus. The apparatus can be equipped for this purpose with a light source, a loudspeaker, or a screen. Depending on the application, it is of course also conceivable for the user to be informed, in the context of a conformity, that the quality of the reagents is still sufficient.

According to a further advantageous embodiment of the invention, the threshold value is stipulated with a user-defined relationship to the reference data. In this case the user defines the threshold value, and thus stipulates the reagent quality that is still sufficient for his or her application.

According to a further advantageous embodiment of the invention, at least one sensor for the detection of electromagnetic radiation, radioactive radiation, optical density, fluorescence, or enzymatic activity is used. Characteristic properties of the test material that are caused by the treatment are sensed with the at least one sensor. Different radiation detectors can be used, including, inter alia, sensors such as photocells, which are suitable for the detection of electromagnetic radiation having a wavelength from near infrared light to UV light, or photomultipliers, which represent particularly highly sensitive detectors in that wavelength region. Geiger-Müller tubes, which serve for the detection of any radioactive radiation, can also be used.

According to a further advantageous embodiment of the invention, what is used as a sensor for the detection of electromagnetic radiation is, in particular, a charge-coupled device (CCD) chip, a complementary metal oxide semiconductor (CMOS) sensor, or a lateral buried charge accumulator and sensing transistor array (LBCAST) sensor, preferably the sensor of a densitometer. The treatment of many cytological or histological prepared specimens concludes with a staining step in which one or more optically detectable products are formed. The objective of the staining actions is to make important structures optically distinguishable, or to visibly detect cell or tissue constituents of interest, by means of different colors. The different dyes emit electromagnetic radiation that is sensed by the various optical sensors. For quantitative measurement of the color density, densitometers can be used for transmitted-light measurements by sensing transmissivity, and for reflected-light measurements by sensing scattering and reflection.

According to a further advantageous embodiment of the invention, a basket, a transport magazine, a specimen slide, a film, a plastic plate, or a textile fabric is used as a carrier element. In order to obtain informative measured values in the form of characteristic properties of the test material that are caused by the treatment, it is necessary for the carrier elements to pass through the individual treatment stations together with the specimens to be treated. One possibility for linking the carrier elements to the specimens involves transporting the specimens to the individual treatment stations in baskets or in transport magazines, the basket or transport magazine serving as a carrier element for the reference medium. Further possibilities involve arranging the test material on a specimen slide made of glass, or on a plastic plate. The specimen slide that is equipped, as a carrier element, with the test material is then arranged, together with the specimens to be treated, in a transport magazine or another specimen holding device. It is also possible to use small frames, over which a film or a textile fabric is stretched, in such a transport magazine or specimen holding device. A film carrying the test material can in turn be mounted, as a carrier element, on a basket or a specimen holding device, thus enabling easy replacement of the carrier element having test material in the context of baskets or specimen holding devices.

According to a further advantageous embodiment of the invention, a biological, organic, inorganic, or synthetic material is used as a test material. If synthetic material is used, it is particularly advantageous that the synthetic material can be manufactured exactly in accordance with the user's needs. Synthetic materials are also, as a rule, stronger than natural materials. Suitable test materials specifically form a detectable product by reaction with one or more reagents that are to be detected. It may furthermore be advantageous, if applicable, for the test material to be incorporated into an embedding medium. Suitable embedding media are, for example, paraffin, waxes, and synthetic resins.

According to a further advantageous embodiment of the invention, at least one cell or at least one tissue section is used as a test material. When cytological or histological test material is used, the advantage exists that this material behaves exactly like the prepared specimens.

According to a further advantageous embodiment of the invention, proteins, proteids, polypeptides, peptides, amino acids, antigens, haptens, epitopes, cytoplasmic proteins, hemoglobin, collagen, nucleic acids, nucleotides, nucleosides, carbohydrates, proteoglycans, sulfated glycosamine glycans, lipids, fatty acids, and modifications of the aforesaid molecules, and combinations, mixtures, conjugates, or fusions of the molecules, are used as a test material. Among the reactions of the test materials with the reagents are electrostatic interactions and chemical reactions. Test material that possesses an anionic nature or a cationic nature can be used, for example, in order to ascertain reagent quality in the context of standard histological stains. Anionic test materials react with cationic dyes. Included among the test materials that have an anionic, i.e. acid, nature are, inter alia, nucleic acids, proteins having many negatively charged groups, and sulfated glycosamine glycans. Included among the basic test materials are a variety of cytoplasmic proteins or hemoglobin. Once the staining treatment is complete, the dye remains bound via electrostatic interactions to the test material that is immobilized on the carrier element. Immobilization of the test materials onto the carrier elements is accomplished using known methods.

According to a further advantageous embodiment of the invention, dyes, metal ions, synthetic polymers, in particular polymers having ionizable or ionic groups or ion-containing polymers, are used as a test material. Many of the reagents that are used in the individual treatment stations are colorless. Their quality can be ascertained by using dyes, immobilized on carrier elements, that are modified by binding of the reagent or reagents in such a way that the wavelength or wavelength region of the radiation absorbed by them changes. Included among these dyes are also those that are colorless before reacting, and whose reaction with the reagent or reagents results in a colored product. In addition, metal ions form complexes with many organic molecules. Some of these complexes are colored, and are therefore suitable for the detection of reagent quality. Synthetic polymers are also suitable for ascertaining reagent quality. They can be manufactured, for example, in suitable manufacturing processes in such a way that they exhibit appropriately charged groups. Semisynthetic polymers that are obtained from natural polymers by the attachment, exchange, or removal of chemical groups, atoms, or charge carriers can also be selected as a test material.

According to a further advantageous embodiment of the invention, replacement and/or metering of the treatment stations is controlled using the evaluation device.

The apparatus for ascertaining reagent quality in the context of units having multiple treatment stations for the treatment of specimens, in particular of cytological and histological prepared specimens, comprises a carrier element having at least one test material, and an evaluation device for evaluating the test material. The carrier element having the test material is conveyed through all the treatment stations through which the specimens also pass. Processing of the test material through the treatment stations occurs simultaneously with the specimens, and is thus the test material is subjected to the same conditions as the specimens. During said processing, the test material reacts with the reagent or reagents of the treatment stations. At least one detectable product is formed in this context. The evaluation device senses the detectable product after the last treatment station.

According to a further advantageous embodiment of the invention, the apparatus comprises a reference carrier element having test material. Treatment of the reference carrier element can occur together with the specimens to be treated. Also conceivable is a separate treatment of the reference carrier element in the treatment stations according to the specimen treatment sequence. After the last treatment station, the characteristic properties of the test material of the reference carrier element that are caused by the treatment are sensed and are stored as reference data. For evaluation of a test material of a carrier element, after the last treatment station the characteristic properties of the test material that are caused by the treatment are evaluated by the evaluation device and compared with the reference data. The test material of the reference carrier material is, as a rule, a test material identical to the test material of the carrier element.

If the characteristic properties of the test materials after passing through the treatment stations are comparable, it is also possible to use different test materials. Upon evaluation, a predefined threshold value stored in the evaluation device is taken into consideration in checking whether a conformity exists between the characteristic properties of the test material and the reference data. It is possible for the threshold value to be stipulated, in this context, with a user-defined relationship to the reference data. Lastly, the user can be informed when a conformity does not exist between the characteristic properties of the test material and the reference data, in consideration of the reference value. The unit can be equipped for this purpose in such a way that the user is informed of this event by the illumination of an indicator light or the emission of a sound.

According to a further advantageous embodiment of the invention, the evaluation device of the apparatus according to the present invention comprises at least one sensor for the detection of electromagnetic radiation, radioactive radiation, optical density, fluorescence, or enzymatic activity. The characteristic properties of the test material that are caused by the treatment are sensed with at least one sensor. Optical sensors such as photocells or photomultipliers can serve, for example, as sensors. Geiger-Müller tubes can furthermore be used.

According to a further advantageous embodiment of the invention, the sensor for the detection of electromagnetic radiation is, in particular, a CCD chip, a CMOS sensor, an LBCAST sensor, preferably the sensor of a densitometer. The treatment of many cytological or histological prepared specimens concludes with a staining step in which one or more optically detectable products are formed. For quantitative measurement of the color density, densitometers can be used, for example, for transmitted-light measurements by sensing transmissivity, and for reflected-light measurements by sensing scattering and reflection.

According to a further advantageous embodiment of the invention, a basket, a transport magazine, a specimen slide, a film, a plastic plate, or a textile fabric is provided as a carrier element of the apparatus. It is also possible to use small frames, over which a film or a textile fabric is stretched, in a transport magazine or another specimen holding device. Carrier elements having test material can furthermore be mounted on a basket or a specimen holding device for easy replacement of the carrier element.

According to a further advantageous embodiment of the invention, a biological, organic, inorganic, or synthetic material is provided as a test material of the apparatus. It may further be advantageous, if applicable, for the test material to be incorporated into an embedding medium. Paraffin, waxes, and synthetic resins are suitable, for example, as embedding media.

According to a further advantageous embodiment of the invention, at least one cell or at least one tissue section is provided as a test material of the apparatus.

According to a further advantageous embodiment of the invention, proteins, proteids, polypeptides, peptides, amino acids, antigens, haptens, epitopes, cytoplasmic proteins, hemoglobin, collagen, nucleic acids, nucleotides, nucleosides, carbohydrates, proteoglycans, sulfated glycosamine glycans, lipids, fatty acids, and modifications of the aforesaid molecules, and combinations, mixtures, conjugates, or fusions of the molecules, are used as a test material of the apparatus. Of the test materials listed above, nucleic acids, proteins having many negatively charged groups, and sulfated glycosamine glycans, among others, possess an anionic, i.e. acid nature. A variety of cytoplasmic proteins or hemoglobin, conversely, are among the basic molecules. In the context of staining treatments that exploit the ionic nature of the target molecules, the dye that is used remains bound via electrostatic interactions to the test material that is immobilized on the carrier element. Immobilization of the test materials onto the carrier elements is accomplished using known methods.

According to a further advantageous embodiment of the invention, dyes, metal ions, natural or synthetic polymers, in particular polymers having ionizable or ionic groups or ion-containing polymers, are used as a test material of the apparatus. Dyes immobilized on carrier elements can be utilized for the detection of colorless reagents. Reaction with the reagent or reagents causes a change in the absorption behavior of the dyes, which is expressed as a color shift. In addition, metal ions can also be provided. These form colored complexes with certain organic molecules.

Further advantages and advantageous embodiments of the invention may be gathered from the description that follows, from the drawings, and from the claims.

### BRIEF DESCRIPTION OF THE DRAWINGS

The drawings depict an exemplifying embodiment of an apparatus according to the present invention. In the drawings:

FIG. 1 is a front view of an apparatus;

FIG. 2 is a view from the side of the apparatus in accordance with FIG. 1;

FIG. 3 shows a specimen slide magazine, having multiple specimen slides, of the apparatus in accordance with FIG. 1;

FIG. 4 shows a computer and sensor of the apparatus in accordance with FIG. 1;

FIG. 5 is a view from above of a carrier element having test material;

FIG. 6 is a view from above of a reference carrier element having test material.

### DETAILED DESCRIPTION OF THE DRAWINGS

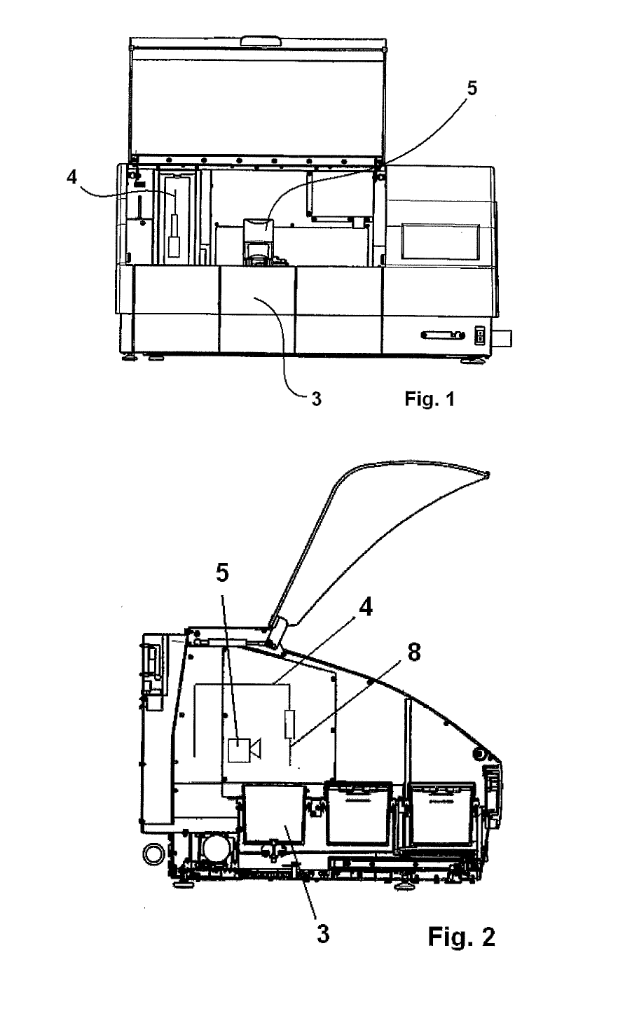

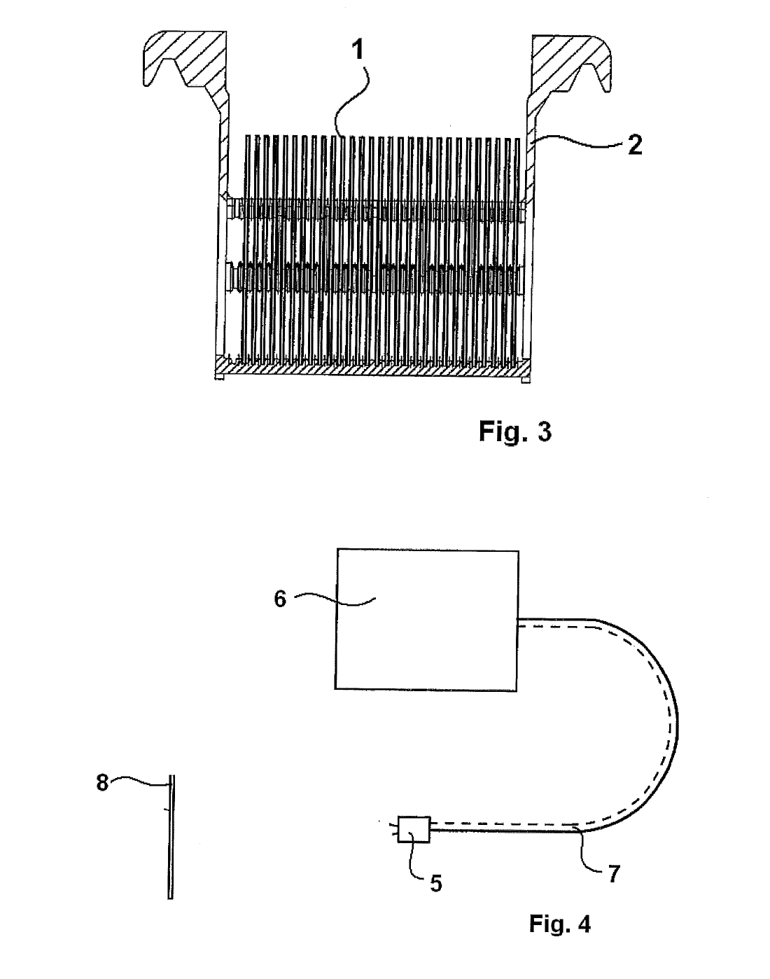

FIGS. 1 and 2 depict an apparatus for the treatment of specimens, in particular of cytological and histological prepared specimens, the specimens being arranged on specimen slides 1. Specimen slides 1 are arranged in specimen slide magazines 2 for treatment in the apparatus. The arrangement of multiple specimen slides 1 in a specimen slide magazine is depicted in FIG. 3. The apparatus according to FIGS. 1 and 2 comprises multiple treatment stations 3 into which the specimens arranged on specimen slides 1 are introduced according to a predefined treatment program. Treatment stations 3 are located next to one another in the apparatus. They are filled with different treatment baths. A transport device 4 is arranged in the apparatus above the treatment stations. It engages onto specimen slide magazines 2 from above, picks up the specimen slide magazines, and transports them from one treatment station 3 to the next. In addition, transport device 4 picks up a specimen slide magazine at the beginning of a treatment in order to introduce it into first treatment station 3. Transport device 4 furthermore conveys a specimen slide magazine out of the apparatus after the treatment is complete and the last treatment station has been left.

The apparatus is equipped with an evaluation device that comprises an optical sensor 5, a computer 6, and a data line 7. Data line 7 connects sensor 5 and computer 6 to one another for data exchange. This is depicted in FIG. 4.

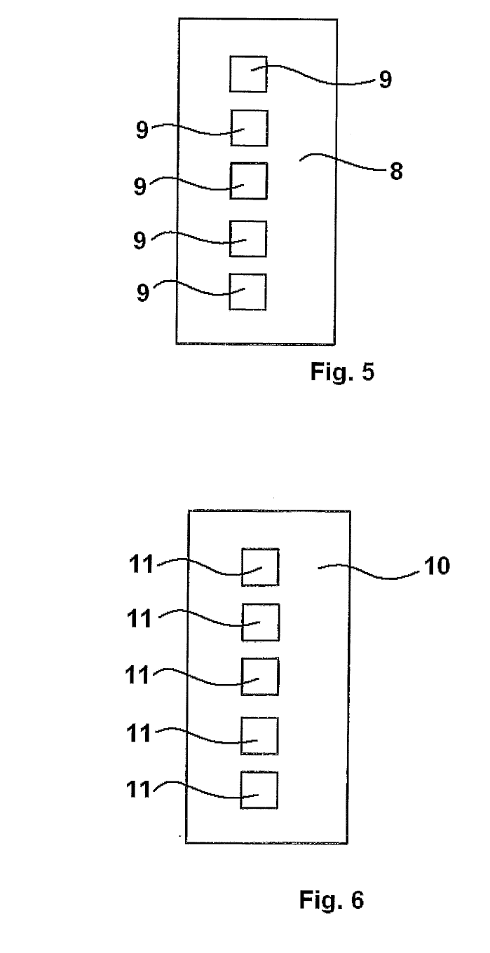

FIG. 5 depicts a carrier element 8 having five regions of multiple test materials 9. A specimen slide serves as a carrier element, so that carrier element 8 can be inserted into specimen slide magazine 2 together with specimen slides 1.

A reference carrier element 10 is depicted in FIG. 6. Exactly like carrier element 8 in FIG. 5, is comprises five regions 11 having multiple test materials. The test materials of carrier element 8 and of reference carrier element 10 conform to one another. The positions of the relevant regions on carrier element 8 and on reference carrier element 10 are likewise identical.

After the last treatment station of a predefined treatment program, regions 9 of the test materials of carrier element 8 are optically sensed by means of sensor 5, and the data acquired in that context are compared with data of reference carrier element 10 that are stored in computer 6. The result of this comparison provides information as to the quality of the reagents in the treatment stations.

All the features, both individually and in any combination with one another, may be essential to the invention.

### LIST OF REFERENCE NUMERALS

1 Specimen slide
2 Specimen slide magazine
3 Treatment station
4 Transport device
5 Sensor
6 Computer
7 Data line
8 Carrier element
9 Region of test material
10 Reference carrier element
11 Region of test material

What is claimed is:

1. A method for determining reagent quality in a device having multiple treatment stations for the treatment of at least one of cytological and histological specimens, the method comprising:
a) providing a carrier element that comprises at least one test material;
b) transferring and treating the carrier element having test material according to a predefined sequence in a plurality of the treatment stations together with the specimens;
c) evaluating the test material by means of an evaluation device after treatment in the last treatment station in sequence.

2. The method according to claim 1, further comprising as a previous method step treating a reference carrier element having a test material by the reagents of the treatment stations in the predefined sequence; and after treatment in the last treatment station in sequence, determining the characteristic properties of the test material that are due to the treatment and storing these characteristic properties as reference data.

3. The method according to claim 2, further comprising comparing for evaluation of the test material in accordance with step c) the characteristic properties of the test material due to the treatment with the reference data.

4. The method according to claim 3, further comprising predefining at least one threshold value and storing it in the evaluation device; and for evaluation of a test material in accordance with step c) checking under taking the threshold value into account whether a similarity exists between the characteristic properties of the test material and the reference data.

5. The method according to claim 4, further comprising informing a user when a similarity does not exist between the characteristic properties of the test material and the reference data under taking the threshold value into account.

6. The method according to claim 4, further comprising setting of the threshold value in a user-defined relationship to the reference data.

7. The method according to claim 1, further comprising reading at least one of electromagnetic radiation, radioactive radiation, optical density, fluorescence, or enzymatic activity by means of at least one sensor.

8. The method according to claim 7, further comprising providing a sensor comprising at least one of a CCD chip, a CMOS sensor, an LBCAST sensor and the sensor of a densitometer.

9. The method according to claim 1, further comprising providing at least one of a basket, a transport magazine, a specimen slide, a film, a plastic plate, and a textile fabric as a carrier element.

10. The method according to claim 1, further comprising providing at least one of a biological, organic, inorganic, and a synthetic material as a test material.

11. The method according to claim 10, further comprising providing at least one of a cell and a tissue section as a test material.

12. The method according to claim 10, further comprising providing at least one of proteins, proteids, polypeptides, peptides, amino acids, antigens, haptens, epitopes, cytoplasmic proteins, hemoglobin, collagen, nucleic acids, nucleotides, nucleosides, carbohydrates, proteoglycans, sulfated glycosamine glycans, lipids, fatty acids, and modifications of the aforesaid molecules, and combinations, mixtures, conjugates, or fusions of the molecules as a test material.

13. The method according to claim 10, further comprising providing at least one of dyes, metal ions, synthetic polymers, polymers having ionizable groups, polymers having ionic groups and ion-containing polymers as a test material.

14. The method according to claim 1, further comprising controlling at least one of replacing and metering of the treatment stations by using the evaluation device.

15. An apparatus for determining reagent quality in a device having multiple treatment stations for the treatment of at least one of cytological and histological specimens, the apparatus comprising:
a carrier element,
at least one test material carried by the carrier element, and
an evaluation device for evaluating the test material.

16. The apparatus according to claim 15, wherein the apparatus comprises a reference carrier element having test material.

17. The apparatus according to claim 15, wherein the evaluation device comprises at least one sensor for reading at least one of electromagnetic radiation, radioactive radiation, optical density, fluorescence, and enzymatic activity.

18. The apparatus according to claim 17, wherein the sensor for reading of electromagnetic radiation is at least one of a CCD chip sensor, a CMOS sensor, an LBCAST sensor, and a densitometer sensor.

19. The apparatus according to one of claims 15, further comprising as a carrier element at least one of a basket, a transport magazine, a specimen slide, a film, a plastic plate, and a textile fabric.

20. The apparatus according to claim 15, wherein the test material is at least one of a biological, organic, inorganic, and synthetic material.

21. The apparatus according to claim 15, wherein the test material comprises at least one of a cell and a tissue section.

22. The apparatus according to claim 20, wherein the test material comprises at least one of proteins, proteids, polypeptides, peptides, amino acids, antigens, haptens, epitopes, cytoplasmic proteins, hemoglobin, collagen, nucleic acids, nucleotides, nucleosides, carbohydrates, proteoglycans, sulfated glycosamine glycans, lipids, fatty acids, and modifications of the aforesaid molecules, and combinations, mixtures, conjugates, or fusions of the molecules.

23. The apparatus according to claim 20, wherein the test material comprises at least one of dyes, metal ions, synthetic polymers, polymers having ionizable groups, polymers having ionic groups and ion-containing polymers.
